(19) United States
(12) Patent Application Publication
(10) Pub. No.: US 2006/0058359 A1
(43) Pub. Date: Mar. 16, 2006

(54) Compounds useful as pesticides

(76) Inventor: Jack Geno Samaritoni, Avon, IN (US)

Correspondence Address: DOW AGROSCIENCES LLC, 9330 ZIONSVILLE RD, INDIANAPOLIS, IN, 46268, US

(21) Appl. No.: 10/535,953

(22) PCT Filed: Dec. 19, 2003
(86) PCT No.: PCT/US03/40737

§ 371 Date: May 20, 2005

Related U.S. Application Data
(60) Provisional application No. 60/435,932, filed on Dec. 20, 2002.

(51) Int. Cl. A01N 43/40; A01N 43/36; C07D 213/53

(52) U.S. Cl. 514/357; 514/408; 548/561; 546/332

(57) ABSTRACT

Compounds useful to control pests are provided.

### PRIORITY

This application claims priority from U.S. provisional application 60/435,932 which was filed on Dec. 20, 2002.

### FIELD OF THE INVENTION

This invention provides compounds that are useful as pesticides.

### BACKGROUND OF THE INVENTION

There is an acute need for new pesticides. For example, insects and mites are developing resistance to the insecticides and acaricides in current use. At least 400 species of arthropods are resistant to one or more insecticides. The development of resistance to some of the older insecticides, such as DDT, the carbamates, and the organophosphates, is well known. But resistance has even developed to some of the newer pyrethroid insecticides and acaricides. Therefore, a need exists for new insecticides and acaricides, and particularly for compounds that have new or atypical modes of action.

### DETAILED DESCRIPTION OF THE INVENTION

[image]

In Figure One Q, R¹, R², R³, R⁴, and R⁵ have the following meanings.

Q can be any five or six membered carbocyclic or heterocyclic ring, such as, phenyl, pyridyl, pyridazinyl, pyrimidinyl, pyrazinyl, pyrrolyl, pyrazolyl, imidazolyl, thienyl, furanyl, thiazolyl, isothiazolyl, oxazolyl, and isoxazolyl, and including reduced forms of the heterocyclic rings such as tetrahydrofuranyl.

R¹, R², R³, R⁴, and R⁵ each independently can be:
(a) a C₁₋₁₀, branched or unbranched, alkyl, alkoxy, alkenyl, alkynyl, alkylthio, alkylsulfinyl, alkylsulfonyl, alkylcarbonyl, alkylcarbonothioyl, alkoxycarbonyl, alkylthiocarbonyl, alkoxycarbonothioyl, alkylthiocarbonothioyl, or HC(═NH)—;
(b) a C₃₋₁₀, cycloalkyl, or cycloalkenyl;
(c) aryl, heterocyclyl, aryloxy, heterocyclyloxy, arylthio, heterocyclylthio, arylamino, or heterocyclylamino; or
(d) a hydro, hydroxy, mercapto, amino, cyano, formyl, nitro, halo, or aminocarbonyl.

Each member of Q, each member of R¹, R², R³, R⁴, and R⁵, which may have a hydrogen atom in a certain position, may instead of having such hydrogen atom, have a:
(a) a C₁₋₁₀, branched or unbranched, alkyl, alkoxy, alkenyl, alkynyl, alkylthio, alkylsulfinyl, alkylsulfonyl, alkylcarbonyl, alkylcarbonothioyl, alkoxycarbonyl, alkylthiocarbonyl, alkoxycarbonothioyl, alkylthiocarbonothioyl, HC(═NH)—, dialkylphosphonyl, or dialkylphosphatyl;
(b) a C₃₋₁₀, cycloalkyl, or cycloalkenyl;
(c) an aryl, heterocyclyl, aryloxy, heterocyclyloxy, arylthio, heterocyclylthio, arylamino, or heterocyclylamino; or
(d) a hydro, hydroxy, mercapto, amino, cyano, formyl, nitro, halo, or aminocarbonyl;
   in such position, provided that these substituents are sterically compatible and the rules of chemical bonding and strain energy are satisfied. Figure One is a generic structure. It should be noted that this generic structure can represent, depending on the substituents used, a number of generic enaminoamidines or iminoamidines due to the presence of the two double bonds. These generic enaminoamidines or iminoamidines can exist in a dynamic equilibrium with each other and so interconvert through various tautomeric or canonical forms by free rotation around the relevant bonds. This invention comprises all such interconverting enaminoamidines or iminoamidines and purified compositions thereof. The nature of tautomeric and canonical forms is understood to be as described in “Advanced Organic Chemistry: Reactions, Mechanisms and Structure”, 4^{th} edition, J. March ed., John Wiley and Sons, New York, 1992.

The term “aryl” means a monovalent radical derived by loss of hydrogen from an aromatic hydrocarbon. The term heterocyclyl means a monovalent radical derived by loss of a hydrogen from an ring structure, where such ring structure contains one or more nitrogen, oxygen, or sulfur atoms. Examples of aryls and heterocyclyl include, but are not limited to, phenyl, naphthyl, pyridyl, pyridazinyl, pyrimidinyl, pyrazinyl, pyrrolyl, pyrazolyl, imidazolyl, thienyl, furanyl, thiazolyl, isothiazolyl, oxazolyl, and isoxazolyl, and included are reduced forms of the heterocyclyl such as tetrahydrofuranyl.

All salts and esters of these compounds are contemplated as part of this invention.

The compounds of the invention are useful for the control pests such as, insects, mites, and aphids. Therefore, the present invention also is directed to a method for inhibiting an insect, mite, or aphid which comprises applying to a locus of the insect or mite an insect- or mite-inhibiting amount of a compound of this invention. In particular, these compounds control insects in the order Homoptera, including the families Aphididae (aphids), Aleyrodidae (whiteflies), Delphacidae (planthoppers), and Cicadellidae (leafhoppers). They also control insects in the order Coleoptera (beetles), including the family Chrysomelidae (leaf beetles). In particular, other representative pests which may be controlled by the method of this invention include members of the Arthropoda, including mites of the suborders Mesostigmata, Sarcoptiformes, Trombidiformes and Onchychopalpida; sucking and biting lice of the orders Anoplura and Mallophaga: ticks of the families Ixodidae and Argasidae: fleas of the families Pulicidae, Ceratophyllidae, and others; Cimex and other Hemiptera; Triatoma and other Heteroptera: and myiasis-related fly larvae and blood sucking adults (including mosquitoes) of the suborders Brachycera, Cyclorrhapha and Nematocera. Representative also are helminths included in the Nematoda (Strongylida, including but not limited to Strongyloidea, Ancylostomatoidea, Trichostrongyloidea and Metastrongyloidea; Ascarida <Ascaris; Filariina, such as but not limited to Onchocerca and Dirofilaria; Rhabditida; and Trichinellida); Cestoidea, especially Cyclophyllidea, and Trematoda, including Strigeatoidea such as Schistosoma; Echinostomida such as Fasciola: and Plagiorchiida such as Paraqonimus. Other pests which may be controlled by compounds of this invention Acanthocephala such as Macracanthorhynchus, Onicola or Moniliformis, and Pentastomida, especially Linguatula; and Protozoa, especially Coccidia such as Eimeria and Plasmodium, Piroplasmea such as Babesia; Toxoplasmea such as Trypanosoma; Trichomonadidae such as Trichomonas and Entamoebidae such as Entamoeba. Illustrative of specific pests of various animals which may be controlled by the method of this invention include arthropods such as mites (mesostigmatids, itch. mange, scabies. chiggers), ticks (soft-bodied and hard-bodied), lice (sucking, biting), fleas (dog flea, cat flea, oriental rat flea), true bugs (bed bugs, kissing bugs), bloodsucking adult flies (horn fly, horse fly, stable fly. black fly. deer fly, louse fly, tsetse fly, punkies, mosquitoes). and parasitic fly maggots (bot fly, blow fly, screwworm, cattle grub, fleeceworm); helminths such as nematodes (threadworm, lungworm, hookworm, whipworm, nodular worm. stomach worm, round worm, pinworm, heartworm), cestodes (tapeworms) and trematodes (liver fluke, blood fluke); protozoa such as coccidia, trypanosomes, trichomonads, amoebas and plasmodia; acanthocephalans such as thomy-headed worms; and pentastomids such as tongueworms.

The compounds are useful for reducing populations of insects and mites and are useful in a method of inhibiting an insect or mite population which comprises applying to a locus of the insect or mite an effective insect- or mite-inactivating amount of a compound of this invention.

The “locus” of insects or mites is a term used herein to refer to the environment in which the insects or mites live or where their eggs are present, including the air surrounding them, the food they eat, or objects or materials which they contact. For example, plant-ingesting insects or mites can be controlled by applying the active compound to plant parts that the insects or mites eat, particularly the foliage. Soil-inhabiting insects such as termites can be controlled by applying the active compound to the soil that the insects move through. Insects such as fleas that infest animals can be controlled by applying the active compound to the animal that is infested. Oral administration of the compounds of this invention may be performed by mixing the compound in the animal's feed or drinking water, vitamin or mineral supplement, or by administering oral dosage forms such as drenches, tablets, bolus, salt block or capsules.

It is contemplated that the compounds might also be useful to protect textiles, paper, stored grain, or seeds by applying an active compound to such substance.

The term “inhibiting an insect or mite” refers to a decrease in the numbers of living insects or mites, or a decrease in the number of viable insect or mite eggs. The extent of reduction accomplished by a compound depends, of course, upon the application rate of the compound, the particular compound used, and the target insect or mite species. At least an inactivating amount should be used.

The terms “insect-inactivating amount” and “mite-inactivating amount” are used to describe the amount, which is sufficient to cause a measurable reduction in the treated insect or mite, population. Generally an amount in the range from about 1 to about 1000 ppm by weight active compound is used. In another embodiment, the present invention is directed to a method for inhibiting a mite or insect which comprises applying to a plant an effective mite- or insect-inactivating amount of a compound of this invention.

The compounds of this invention are applied in the form of compositions which comprise a compound of this invention and a phytologically-acceptable inert carrier. The compositions are either concentrated formulations which are dispersed in water for application, or are dust or granular formulations which are applied without further treatment. The compositions are prepared according to procedures and formulae which are conventional in the agricultural chemical art, but which are novel and important because of the presence therein of the compounds of this invention.

The dispersions in which the compounds are applied are most often aqueous suspensions or emulsions prepared from concentrated formulations of the compounds. Such water-soluble, water-suspendable or emulsifiable formulations are either solids, usually known as wettable powders, or liquids usually known as emulsifiable concentrates or aqueous suspensions. Wettable powders, which may be compacted to form water dispersible granules, comprise an intimate mixture of the active compound, an inert carrier, and surfactants. The concentration of the active compound is usually from about 10% to about 90% by weight. The inert carrier is usually chosen from among the attapulgite clays, the montmorillonite clays, the diatomaceous earths, or the purified silicates.

Effective surfactants, comprising from about 0.5% to about 10% of the wettable powder, are found among the sulfonated lignins, the condensed naphthalenesulfonates, the naphthalenesulfonates, the alkylbenzene-sulfonates, the alkyl sulfates, and nonionic surfactants such as ethylene oxide adducts of alkyl phenols.

Emulsifiable concentrates of the compounds comprise a convenient concentration of a compound, such as from about 50 to about 500 grams per liter of liquid, equivalent to about 10% to about 50%, dissolved in an inert carrier which is either a water miscible solvent or a mixture of water-immiscible organic solvent and emulsifiers. Useful organic solvents include aromatics, especially the xylenes, and the petroleum fractions, especially the high-boiling naphthalenic and olefinic portions of petroleum such as heavy aromatic naphtha. Other organic solvents may also be used, such as the terpenic solvents including rosin derivatives, aliphatic ketones such as cyclohexanone, and complex alcohols such as 2-ethoxyethanol. Suitable emulsifiers for emulsifiable concentrates are chosen from conventional nonionic surfactants, such as those discussed above.

Aqueous suspensions comprise suspensions of water-insoluble compounds of this invention, dispersed in an aqueous vehicle at a concentration in the range from about 5% to about 50% by weight. Suspensions are prepared by finely grinding the compound, and vigorously mixing it into a vehicle comprised of water and surfactants chosen from the same types discussed above. Inert ingredients, such as inorganic salts and synthetic or natural gums, may also be added, to increase the density and viscosity of the aqueous vehicle. It is often most effective to grind and mix the compound at the same time by preparing the aqueous mixture, and homogenizing it in an implement such as a sand mill, ball mill, or piston-type homogenizer.

The compounds may also be applied as granular compositions, which are particularly useful for applications to the soil. Granular compositions usually contain from about 0.5% to about 10% by weight of the compound, dispersed in an inert carrier which consists entirely or in large part of clay or a similar inexpensive substance. Such compositions are usually prepared by dissolving the compound in a suitable solvent and applying it to a granular carrier which has been pre-formed to the appropriate particle size, in the range of from about 0.5 to 3 mm. Such compositions may also be formulated by making a dough or paste of the carrier and compound and crushing and drying to obtain the desired granular particle size.

Dusts containing the compounds are prepared simply by intimately mixing the compound in powdered form with a suitable dusty agricultural carrier, such as kaolin clay, ground volcanic rock, and the like. Dusts can suitably contain from about 1% to about 10% of the compound.

The active compositions may contain adjuvant surfactants to enhance deposition, wetting and penetration of the compositions onto the target crop and organism. These adjuvant surfactants may optionally be employed as a component of the formulation or as a tank mix. The amount of adjuvant surfactant will vary from 0.01 percent to 1.0 percent v/v based on a spray-volume of water, preferably 0.05 to 0.5 percent. Suitable adjuvant surfactants include ethoxylated nonyl phenols, ethoxylated synthetic or natural alcohols, salts of the esters of sulphosuccinic acids, ethoxylated organosilicones, ethoxylated fatty amines, crop oil concentrates containing high molecular weight paraffinic oils and blends of surfactants with mineral and vegetable oils.

It is equally practical, when desirable for any reason, to apply the compound in the form of a solution in an appropriate organic solvent, usually a bland petroleum oil, such as the spray oils, which are widely used in agricultural chemistry.

Insecticides and acaricides are generally applied in the form of a dispersion of the active ingredient in a liquid carrier. It is conventional to refer to application rates in terms of the concentration of active ingredient in the carrier. The most widely used carrier is water.

The compounds of the invention can also be applied in the form of an aerosol composition. In such compositions the active compound is dissolved or dispersed in an inert carrier, which is a pressure-generating propellant mixture. The aerosol composition is packaged in a container from which the mixture is dispensed through an atomizing valve. Propellant mixtures comprise either low-boiling halocarbons, which may be mixed with organic solvents, or aqueous suspensions pressurized with inert gases or gaseous hydrocarbons.

The actual amount of compound to be applied to loci of insects, mites, and aphids is not critical and can readily be determined by those skilled in the art in view of the examples above. In general, concentrations of from-10 ppm to 5000 ppm by weight of compound are expected to provide good control. With many of the compounds, concentrations of from 100 to 1500 ppm will suffice.

The locus to which a compound is applied can be any locus inhabited by an insect or arachnid, for example, vegetable crops, fruit and nut trees, grape vines, and ornamental plants.

Because of the unique ability of mite eggs to resist toxicant action, repeated applications may be desirable to control newly emerged larvae, as is true of other known acaricides.

In addition to being effective against mites, aphids, and insects when applied to foliage, compounds of this invention have systemic activity. Accordingly, another aspect of the invention is a method of protecting a plant from insects which comprises treating plant seed prior to planting it, treating soil where plant seed is to be planted, or treating soil at the roots of a plant after it is planted, with an effective amount of a compound of this invention.

The action of the inventive compounds can be broadened by adding other, for example insecticidally, acaricidally, and/or nematocidally active, ingredients. For example, one or more of the following compounds can suitably be combined with the compounds of the invention:
(1) organophosphorus compounds such as acephate, azinphosmethyl, cadusafos, chlorethoxyfos, chlorpyrifos, coumaphos, dematon, demeton-S-methyl, diazinon, dichlorvos, dimethoate, EPN, erthoate, ethoprophos, etrimfos, fenamiphos, fenitrothion, fensulfothion, fenthion, fonofos, formothion, fosthiazate, heptenophos, malathion, methamidophos, methyl parathion, mevinphos, monocrotophos, parathion, phorate, phosalone, phosmet, phosphamidon, phosphocarb, phoxim, profenofos, propaphos, propetamphos, prothiofos, pyrimiphos-methyl, pyrimiphos-ethyl, quinalphos, sulprofos; tebupirimphos, temephos, terbufos, tetrachlorvinphos, thiafenox, thiometon, triazophos, and trichlorphon;
(2) carbamates such as aldicarb, bendiocarb, benfuracarb, bensultap, BPMC, butoxycarbocim, carbaryl, carbofuran, carbosulfan, cloethocarb, ethiofencarb, fenobucarb, furathiocarb, methiocarb, isoprocarb, methomyl, oxamyl, pirimicarb, promecarb, propoxur, thiodicarb, and thiofurox;
(3) pyrethroids such as acrinathrin, allethrin, beta-cyfluthrin, bifenthrin, bloresmethrin, cyfluthrin; cyhalothrin; lambda-cyhalothrin; gamma-cyhalothrin, cypermethrin; alpha-cypermethrin; zeta-cypermethrin; deltamethrin, esfenvalerate, fenvalerate, fenfluthrin, fenpropathrin, flucythrinate, flumethrin, fluvalinate, tau-fluvalinate, halfenprox, permethrin, protrifenbute, resmethrin, silafluofen, tefluthrin, tetramethrin, tralomethrin, fish safe pyrethroids for example ethofenprox, natural pyrethrin, tetramethrin, s-bioallethrin, fenfluthrin and prallethrin;
(4) acylureas, other types of insect growth regulators and insect hormone analogs such as buprofezin, chromfenozide, chlorfluazuron, diflubenzuron, fenoxycarb, flufenoxuron, halofenozide, hexaflumuron, hydroprene, leufenuron, methoprene, methoxyfenozide, novaluron, pyriproxyfen, teflubenzuron and tebufenozide, N-[3,5-dichloro-2-fluoro-4-(1,1,2,3,3,3-hexafluoropropoxy)phenyl]-N′(2,6-difluorobenzoyl)urea;
(5) neonicotnioids and other nicotinics such as acetamiprid, AKD-1022, cartap, TI-435, clothianidin, MTI-446, dinotefuran, imidacloprid, nicotine, nitenpyram, thiamethoxam, thiacloprid;
(6) macrolides such as avermectins, milbemycins, or spinosyns for example such as abamectin, ivermectin, milbemycin, emamectin benzoate and spinosad; and
(7) other insecticidal, acaricidal, mollscicial and nematocidal compounds or actives such as aldrin, amitraz, azadirachtin, azocyclotin. bifenazate, bromopropylate, chlordimeform, chlorfenapyr, chlofentezine, chlorobenzilate, chlordane, cyhexatin, cyromazin, DDT, dicofol, dieldrin, DNOC, endosulfan, ethoxazole, fenazaquin, fenbutatin oxide, fenproximate, beta-fenpyroximate, fipronil, flubenzimine, hexythiazox, IKI-220, indoxacarb, lindane, methiocarb, metaldehyde, methoxychlor, neem, petroleum and vegetable oils, pyridaben, pymetrozine, pyrimidifen, rotenone, S-1812, S-9539, spirodiclofen, sulfur, tebufenpyrad, tetradifon, triazamate, an insect-active extract from a plant; a preparation containing insect-active nematodes, a preparation obtainable from Bacillus subtilis, Bacillus thuringiensis, a nuclear polyhedrosis virus, or other like organism genetically modified or native, as well as synergists such as piperonyl butoxide, sesamax, safroxan and dodecyl imidazole, and phagostimulants such as cucurbitacin, sugars and Coax.

### EXAMPLES

These examples are provided to further illustrate the invention. They are not meant to be construed as limiting the invention.

### Example One

### Preparation of Compound One

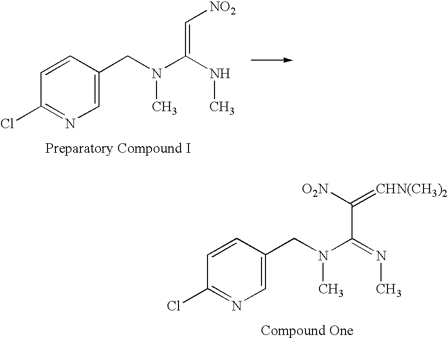

A solution of 500 mg (1.95 mmol) of Preparatory Compound I (Isao, et. al., Synthesis and Insecticidal Activity of Acyclic Nitroethene Compounds Containing a 3-Pyridylmethylamino Group. J. Pestic. Sci., 1993, 18, 31-40) and 3 mL of dimethylformamide dimethylacetal in 3 mL of dry toluene was heated at 105-110° C. for 2 hours and was allowed to cool to room temperature, which was about 22° C. The mixture was concentrated in vacuo to a residue, which was then triturated under ethyl ether to afford 190 mg (31%) of Compound One as a yellowish brown solid.

### Example Two

### Preparation of Compound Two

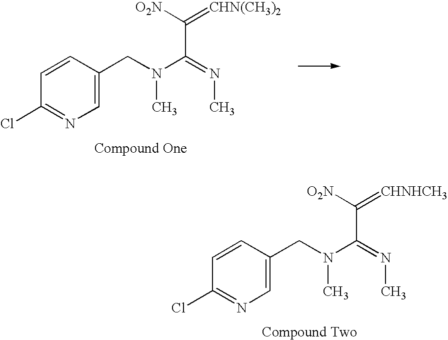

A solution of 130 mg (0.417 mmol) of Compound One and 0.5 mL of 40% aqueous methylamine in 2 mL of methanol was stirred at room temperature, which was approximately 22° C. for 5 hours and was then concentrated to give 110 mg of Compound Two.

### Example Three

### Preparation of Compound Three

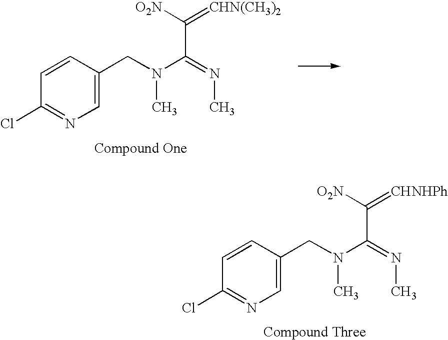

A solution of 200 mg (0.641 mmol) of Compound One, 60 mg (0.64 mmol) of aniline, and 61 mg (0.64 mmol) of methanesulfonic acid was stirred overnight at room temperature, which was approximately 22° C. The solution was concentrated to a residue, which was then chromatographed on silica gel (230-400 mesh) using 95/5 and 90/10 dichloromethane/methanol as eluant to afford 130 mg of Compound Three.

### Example Four

### Testing with Cotton Aphid (Aphis gossypii)

Yellow crookneck squash, Cucurbita pepo, is planted in 3 inch pots and placed in a greenhouse. Plants are watered regularly for 5 to 7 days until they reach the first emergent leaf stage. Plants are then trimmed to a single cotyledon. The squash assay consists of four squash plants per treatment with each plant cotyledon considered a replicate. Four additional plants are used as a control treatment (receiving solvent blank application only). Twenty-four hours prior to application, a leaf section of heavily infested squash plant from the aphid colony is placed onto each cotyledon, allowing a mixed population of A. gossypii nymphs and adults to migrate and infest the test plants. The pre-infested squash cotyledons are sprayed on both the upper and lower surfaces using an airbrush sprayer set at 2 psi. Formulation is an aqueous solution containing 5% solvent and 0.025% Tween 20 surfactant to yield concentrations of 50 ppm and 5 ppm of the test compound. Plants are sprayed to runoff. Tests are held in ambient laboratory temperatures for three days. At 3 days after application (DM) the number of live aphids are counted with the aid of a dissecting microscope. The number of live aphids in the treatment is compared to the number of live aphids in the solvent blank-treated controls and percent mortality is calculated.

Compounds One and Three were tested in this method. The results indicate that both compounds resulted in 100 percent mortality at a rate of 50 and of 5 parts per million.

### Example Five

### Testing with Cat Flea (Cntenocephalides felis)

Media is prepared by mixing 1 part dried bovine blood, 4 parts ground dog food pellets, and 95 parts clean sand. Compounds are dissolved in acetone and further diluted in acetone to yield the required range of concentrations. The acetone solutions are applied to measured amounts of media in small dishes. Final concentrations are expressed as micrograms of compound per gram of media. After the acetone has dried, the treated media is dispensed into small glass vials and infested with 5 to 10 cat flea (Cntenocephalides felis) eggs. The vials are loosely capped and held under controlled conditions for five weeks. Activity is assessed based on the average number of adult cat fleas present in the treated vials compared to the average number of adult cat fleas in the media treated with acetone only.

The results indicate that Compound One showed 90% control of cat fleas at the 0.1 μg/g rate, whereas Compound Three showed 38% control of cat fleas at the 0.1 μg/g rate.

We claim:

1. A compound, and all interconverting equilibrium forms of such compound, according to FIG. 1 of the drawings.
wherein
Q can be any five or six membered carbocyclic or heterocyclic ring
R¹, R², R³, R⁴, and R⁵ each independently can be
(a) a C₁₋₁₀, branched or unbranched, alkyl, alkoxy, alkenyl, alkynyl, alkylthio, alkylsulfinyl, alkylsulfonyl, alkylcarbonyl, alkylcarbonothioyl, alkoxycarbonyl, alkylthiocarbonyl, alkoxycarbonothioyl, alkylthiocarbonothioyl, or HC(═NH)—,
(b) a C₃₋₁₀, cycloalkyl, or cycloalkenyl,
(c) an aryl, heterocyclyl, aryloxy, heterocyclyloxy, arylthio, heterocyclylthio, arylamino, or heterocyclylamino; or
(d) a hydro, hydroxy, mercapto, amino, cyano, formyl, nitro, halo, or aminocarbonyl
and wherein each member of Q, each member of R¹, R², R³, R⁴, and R⁵, which has a hydrogen atom in a certain position, may instead of having such hydrogen atom, have a
(a) a C₁₋₁₀, branched or unbranched, alkyl, alkoxy, alkenyl, alkynyl, alkylthio, alkylsulfinyl, alkylsulfonyl, alkylcarbonyl, alkylcarbonothioyl, alkoxycarbonyl, alkylthiocarbonyl, dialkylphosphonyl, or dialkylphosphatyl,
(b) a C₃₋₁₀, cycloalkyl, or cycloalkenyl.
(c) an aryl, heterocyclyl, aryloxy, heterocyclyloxy, arylthio, heterocyclylthio, arylamino, or heterocyclylamino, or
(d) a hydro, hydroxy, mercapto, amino, cyano, formyl, nitro, halo, or aminocarbonyl
in that position.

2. A composition comprising a compound according to claim 1 and at least one other active compound where such active compound is at least insecticidally, acaricidally, or nematocidally active.

3. A process of applying a compound according to claim 1, or a composition according to claim 2, to a locus in an amount effective to control pests.

4. A process of applying a compound according to claim 1, or a composition according to claim 2, to a locus in an amount effective to control insects or mites.

5. A process of topically applying a compound according to claim 1, or a composition according to claim 2, to an animal in an amount effective to control fleas.

6. A process of orally administering a compound according to claim 1, or a composition according to claim 2, to an animal in an amount effective to control fleas.
